(19) United States
(12) Patent Application Publication
(10) Pub. No.: US 2009/0246196 A1
(43) Pub. Date: Oct. 1, 2009

(54) COMBINED TARGETED THERAPY FOR THE TREATMENT OF PROLIFERATIVE DISEASE

(76) Inventors: Leo Linbeck, III, Houston, TX (US); Martin Lindenberg, Bellaire, TX (US); John J. Nemunaitis, Cedar Hill, TX (US); Neil Senzer, Dallas, TX (US); David Shanahan, Dallas, TX (US)

Correspondence Address: CONLEY ROSE, P.C.;David A. Rose, P. O. BOX 3267, HOUSTON, TX, 77253-3267, US

(21) Appl. No.: 12/282,618

(22) PCT Filed: Mar. 15, 2007
(86) PCT No.: PCT/US07/64078

§ 371 Date: Dec. 23, 2008

Related U.S. Application Data
(60) Provisional application No. 60/782,457, filed on Mar. 15, 2006.

(51) Int. Cl. A61K 39/395; A61K 38/45; A61K 38/16; A61K 31/66; A61K 31/517

(52) U.S. Cl. 424/133.1; 424/94.5; 514/12; 514/120; 514/266.4

(57) ABSTRACT

A method of treating an individual comprising: evaluating the disease state of said individual through quantitative and/or qualitative assays; administering in amounts sufficient to treat said disease at least three agents wherein at least one agent is an inhibitor of the human epidermal receptor pathway, at least one agent is signal transduction inhibitor and at least one agent is an angiogenesis inhibitor and; reevaluating the disease state of said individual presenting through quantitative and/or qualitative assays. A composition comprising a first agent that is an inhibitor of the human epidermal receptor pathway, a second agent that is an angiogenesis inhibitor and a third agent that is an inhibitor of Akt wherein said first agent, second agent, third agent and combinations thereof are present in amounts that when administered to an individual in a diseased state are sufficient to treat said disease.

### STATEMENT REGARDING FEDERALLY SPONSORED RESEARCH OR DEVELOPMENT

Not applicable.

### BACKGROUND OF THE INVENTION

1. Field of the Invention

This disclosure relates to combinations of targeted therapies for the treatment of proliferative diseases such as cancer.

2. Background of the Invention

Cancer is a disease marked by the deregulation of normal cellular processes such as maturation, division and senescence. Cancer cells, typically harboring numerous genetic alterations, may evolve to ever more aggressive growth phenotypes. In some cases, disease progression may involve metastasis or spread of the cancer from a primary location to distal sites via the lymph system or bloodstream.

Traditional cancer treatments are often multimodal utilizing some combination of surgery, radiation and chemotherapy to affect a therapeutic response. The effectiveness of any one or a combination of these treatments is limited by the extent to which the treatment can be employed. For example, chemotherapeutic agents are administered at dosages and schedules that limit their toxicity to normal host cells while eliciting a response from cancer cells. This range, between a therapeutic response and toxicity to the host is referred to as the therapeutic window. The therapeutic window outlines the parameters in which a treatment can effectively operate without causing undue harm to the host. This therapeutic window governs all areas of cancer treatment and is a critical consideration in devising new therapeutic regimens.

Recent advances in cancer therapy have led to the rational development of compounds and methods designed to interfere with specific cellular pathways that promote the growth and/or development of cancer cells. Such compounds and methods are collectively referred to as targeted therapy. Despite the promising results seen with targeted therapy, these therapies have often succumbed to the phenomenon of acquired drug resistance. Acquired drug resistance refers to the ability of a cell that initially was adversely affected by a drug to develop pathways or mechanisms that render the cell refractory to the drug. Acquired drug resistance is a well-known phenomenon whose causes are typically multifactorial. One approach to the problem of acquired drug resistance has been to attempt to identify prior to treatment, variables that may contribute to the development of drug resistance. However, there remains no effective methodology for treating a proliferative disease such as cancer while minimizing or preventing the emergence of drug resistance. Thus, there is an ongoing need to develop methodologies for broadening the therapeutic window of agents designed to treat proliferative diseases and for developing methodologies to overcome acquired drug resistance.

### BRIEF SUMMARY OF SOME OF THE PREFERRED EMBODIMENTS

Disclosed herein is a method of treatment comprising: evaluating the disease state of an individual through quantitative and/or qualitative assays; administering in amounts sufficient to treat said disease at least three agents wherein at least one agent is an inhibitor of the human epidermal receptor pathway, at least one agent is signal transduction inhibitor and at least one agent is an angiogenesis inhibitor and; reevaluating the disease state of said individual presenting through quantitative and/or qualitative assays.

Further disclosed herein is a composition comprising a first agent that is an inhibitor of the human epidermal receptor pathway, a second agent that is an angiogenesis inhibitor and a third agent that is an inhibitor of Akt wherein said first agent, second agent, third agent and combinations thereof are present in amounts that when administered to an individual in a diseased state are sufficient to treat said disease.

The foregoing has outlined rather broadly the features and technical advantages of the present invention in order that the detailed description of the invention that follows may be better understood. Additional features and advantages of the invention will be described hereinafter that form the subject of the claims of the invention. It should be appreciated by those skilled in the art that the conception and the specific embodiments disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### DETAILED DESCRIPTION OF THE PREFERRED EMBODIMENTS

In an embodiment, a method of treatment is disclosed. The treatment may be administered to an individual in a diseased state. An “individual” is a vertebrate, alternatively a mammal, alternatively a human. Mammals include, but are not limited to, farm animals, sport animals, rodents, primates, and pets. As used herein, “treatment” is an approach for obtaining beneficial or desired clinical results. Herein, beneficial or desired clinical results include, but are not limited to, alleviation of symptoms, diminishment of extent of disease, stabilization (i.e., not worsening) of state of disease, prevention of spread (i.e., metastasis) of disease, delay or slowing of disease progression, amelioration or palliation of the disease state, improvement in quality of life, and remission (whether partial or total), whether detectable or undetectable. “Treatment” can also mean prolonging survival as compared to expected survival if not receiving treatment. “Palliating” a disease means that the extent and/or undesirable clinical manifestations of a disease state are lessened and/or time course of the progression is slowed or lengthened, as compared to not administering the treatment disclosed herein.

A method of treatment may comprise evaluating the disease state of an individual, administering in amounts sufficient to treat said disease at least three agents wherein at least one agent is an inhibitor of the human epidermal receptor pathway, at least one agent is a signal transduction inhibitor, and at least one agent is an angiogenesis inhibitor and reevaluating the disease state of said individual. The administration of these agents in combination may beneficially effect the individual and/or generate desired clinical results such as for example the reduction of tumor load in an individual presenting with proliferative disease. Said individual may also have a reduced occurrence of acquired drug resistance.

In one embodiment, the method of treatment comprises treatment of an individual presenting with a proliferative disease. In an embodiment, the method treating an individual with a proliferative disease comprises administration of at least one agent which affects tumor cell proliferation, tumor cell differentiation, tumor cell invasion, tumor cell metastasis or combinations thereof. Herein said agent is denoted Agent X. In an embodiment, Agent X is any compound compatible with the other compounds of this disclosure and able to affect the described function. Alternatively, Agent X is any modified alkylphospholipid, which affects tumor cell proliferation, differentiation, invasion, and metastasis. Without wishing to be limited by theory, Agent X may function to inhibit or otherwise modify signaling through at least one signal transduction pathway including but not limited to the pathways involving Akt, MAPK, and JNK. The effects of Agent X on Akt are of particular interest because of 1) the importance of this pathway in the development of most cancers; 2) the evidence that it is often activated in tumors that are resistant to other forms of anticancer therapy; and 3) and the difficulty encountered thus far in the discovery of drugs that will inhibit this pathway without causing excessive toxicity.

Akt or protein kinase B (PKB) is a well-characterized serine/threonine kinase that acts to promote cell proliferation and survival. Akt is involved in multiple pathways in the signal transduction cascade. Signaling through the phosphatidylinositol-3, kinase (PI3-K)/Akt pathway begins with the activation of membrane-spanning receptor tyrosine kinases (RTKs). Upon activation by ligand, RTKs engage and activate PI3-K, which in turn converts membrane-bound phosphatidylinositol (4,5)-bisphosphonate (PIP2) to phosphatidylinositol (3,4,5)-triphosphonate (PIP3). PIP3 then recruits Akt from the cytoplasm to the plasma membrane, where it undergoes a conformational change and becomes activated by phosphorylation of two residues. When Akt is activated (in which case it becomes pAkt) it inhibits apoptosis through phosphorylation of substrates such as BAD or caspase, enhances cellular proliferation through promotion of the cell cycle, and stimulates insulin signaling.

There are several ways in which Akt can be abnormally or persistently activated in cancer. Ligands for cell-surface receptor tyrosine kinases (or the receptors themselves) that stimulate P13-K/Akt signaling may be abnormally increased in some cancers. The HER2/neu receptor has been shown to activate the Akt pathway in HER2/neu-overexpressing breast cancer cells. Mutations of the tumor suppressor gene PTEN are another important cause of increased Akt activation. PTEN is a phosphatase that antagonizes the kinase function of PI3-K and therefore serves as a negative regulator of Akt activity. Finally, Akt may be constitutively activated because of overexpression of Akt isoforms. Thus far, Akt isoforms have been found to be overexpressed in ovarian, breast, prostate, and pancreatic cancers. Elevated levels of pAkt have been correlated with poor prognosis in patients with gastric, hepatocellular, endometrial (with accompanying loss of PTEN), prostate, renal cell and head and neck cancers, as well as glioblastoma. The majority of tumors expressing pAkt were high-grade, advanced stage or had other features associated with poor prognosis.

In an embodiment, the method treating an individual with a proliferative disease comprises administration of at least one agent designed to target the human epidermal growth factor receptor (HER1) pathway. HER1, also known as epidermal growth factor receptor (EGFR), is a component of the HER signaling pathway, which plays a role in non-small cell lung cancer (NSCLC).

An example of an agent designed to target the HER1 pathway includes without limitation TARCEVA commercially available from OSI pharmaceuticals.

In an embodiment, the method of treating an individual with a proliferative disease comprises administration of at least one agent designed to inhibit Vascular Endothelial Growth Factor (VEGF). VEGF is a protein that plays an important role in tumor angiogenesis and maintenance of existing tumor vessels. In an embodiment, any agent that is a VEGF inhibitor and chemically compatible with the other components of this method may be employed. Alternatively, the VEGF inhibitor is a monoclonal antibody. An example of a suitable VEGF inhibitor includes without limitation AVASTIN commercially available from Genentech Inc. AVASTIN is a recombinant humanized anti-VEGF monoclonal antibody composed of human IgG1 framework regions and antigen-binding complementarity-determining regions from a munne monoclonal antibody (muMAb VEGF A.4.6.1). Approximately 93% of the amino acid sequence, including most of the antibody framework, is derived from human IgG1, and ˜7% of the sequence is derived from the murine antibody.

In an embodiment, a method of treating an individual with a proliferative disease comprises administration of a composition comprising at least one modified alkylphospholipid which affects tumor cell proliferation, differentiation, invasion, and metastasis, at least one inhibitor of the HER1 pathway and at least one inhibitor of VEGF. Alternatively, a method of treating an individual with a proliferative disease comprises administration of a composition comprising AVASTIN, TARCEVA and GLEEVAC.

These compositions may be administered in amounts effective to treat an individual with proliferative disease. As will be understood by one skilled in the art the effective amounts and timing of administration, collectively termed dosing, is dependent on the severity and responsiveness of the condition to be treated, with course of treatment lasting from several days to the duration of the life of the individual. Herein an “effective amount” or “therapeutic amount” is an amount sufficient to effect beneficial or desired clinical results. An effective amount can be administered in one or more administrations. For the purposes disclosed herein, an effective amount of the disclosed drug combinations is an amount that is sufficient to palliate, ameliorate, stabilize, reverse, slow or delay the progression of the disease state.

Optimal dosing schedules and dosing amounts can be calculated by one skilled in the art. For instance, optimal dosages are generally 10 times below the lethal dose. The LD₅₀ (the dose that kills 50% of the test animals) can be determined for the agents disclosed herein as well as for the composition of the present disclosure. After individuals are treated, the dosages can be reduced in amount or frequency if the individual exhibits signs of toxicity or increased if the individual tolerates the dosage regime.

Optimal dosing schedules can also be calculated from measurements of drug accumulation in the body. Persons of ordinary skill in the art can easily determine optimum dosages, dosing methods, and repetition rates. Optimum dosages may vary depending on the potency of the composition, and can generally be estimated based on LD₅₀'s from in vitro studies.

In an embodiment, the method of treating an individual with proliferative disease further comprises evaluation of the disease state of the individual through qualitative and/or quantitative assays. Such evaluations may be performed prior to treatment, during treattnent or after completion of treatment or combinations thereof. Evaluations of the disease state may be qualitative and may comprise assays as known to one of ordinary skill in the art. In an embodiment, the qualitative and/or quantitative assays are used prior to treatment of an individual and are used to establish the presence of a disease state such as for example, a proliferative disease state. In an embodiment, the disease state may be evaluated by routine physical exam of the individual presenting with a proliferative disease. In other embodiments, the disease state may be evaluated through the use of questionnaires regarding the quality of life (QoL) of the individual presenting with proliferative disease. Alternatively, the disease state may be evaluated through the use of qualitative assays such as immunological products that may serve as markers of disease. Such immunological assays are known to one of ordinary skill in the art and as will be understood by one of such skill will depend on the type of proliferative disease being evaluated.

In other embodiments, the disease state may be evaluated through techniques to detect circulating tumor cells. For example, a technique of isolating and enumerating circulating tumor cells (CTC) in individuals with cancer using antibody-coated magnetic beads to enrich the blood sample for cells expressing EpCAM (epithelial cell adhesion molecule) has been evaluated in patients with metastatic breast cancer. The number of CTC, which have been shown to be malignant and derived from the primary tumor, is an independent predictor of progression free survival and overall survival in patients with metastatic breast cancer.

Reports in the recent literature describe techniques of isolation and characterization of these CTCs. The findings that CTCs can be found in patients before the primary tumor is detected, that CTCs are found in a significant proportion of patients when a carcinoma recurs, and that CTCs persist in some patients after removal of the primary tumor (suggestive of subclinical disease or dormancy) have been the impetus for continued evaluation of this technology. Evidence that CTCs are derived from clones in the primary tumor suggests that they may reflect the tumor burden throughout all stages of progression. A particularly important attribute of a blood test is that it is safe and can be performed frequently, insofar as repeated invasive procedures, including bone marrow aspiration, may result in limited patient compliance.

Recently the FDA has approved the use of a novel validated Cell Search system to accurately identify circulating tumor cells using cytokeratin antibodies and immune magnetic technology. Ferrofluids coated with epithelial cell specific EpCAM antibodies are used to immunomagnetically enrich epithelial cells, which are then labeled with the fluorescent nucleic dye 4,2-diamidino-2-phenylindole dihydrochloride. This is mixed with two phycoerythrin-conjugated antibodies that bind cytokeratin 8, 18 and 19 and leukocyte specific CD45-allophycocyan antibodies are used to distinguish CECs from leukocytes. 7.5 ml of blood is mixed into this, centrifuged and placed on a CELL PREP system, which is analyzed by an immunomagnetic device called the MAGNEST Cell Presentation Device (Veridx, Raritan, N.J.). Samples are further analyzed using the Cell System Analyzer.

In an embodiment, the disease state of an individual may be evaluated through quantitative assays. Such evaluations may be performed prior to treatment, during treatment or after completion of treatment. Quantitative techniques for evaluation of the disease state are well known to one of ordinary skill in the art. Examples of quantitative techniques for the evaluation of the disease state include without limitation, pharmacokinetic assays and, pharmacodynamic assays.

Pharmacokinetics herein refers to the study of the time course of substances and their relationship with an organism or system while pharmacodynamics herein refers to the study of the biochemical and physiological effects of drugs and the mechanisms of drug action and the relationship between drug concentration and effect. Examples of assays to assess the pharmacokinetics and pharmacodynamics of treatment with the disclosed agents include but are not limited to immunological assays, mass spectroscopy, magnetic resonance imaging, dynamic computed tomography, positron emission tomography and ultrasound. As would be understood by one of ordinary skill in the art, such assays may be performed on the individual as a whole, or on specimens obtained from individuals such as whole blood cells or tissues from biopsies.

In an embodiment, the disease state of an individual may be evaluated through genomic assays. Genetic and epigenetic perturbations in signal pathways drive cancer growth, survival, invasion and metastatic spread. Evidence is emerging to support the concept that each individual's cancer might have a unique complement of pathogenic molecular derangements. This supplies the rationale to incorporate strategic selection of a therapeutic complex from a menu of targeting options that best match the individual molecular profile of the tumor into the clinical decision making.

In an embodiment, a genomic assay for the evaluation of disease state comprises the use of a microarray. The field of microarray technology has rapidly evolved. Over the last 5 years it has become possible to simultaneously analyze integrity and/or expression level of hundreds of thousands of genes within days. A microarray chip smaller than the size of a stamp can identify and semi-quantitatively characterize the expression level of essentially all known human genes simultaneously. Microarray technology can be used to examine the integrity of genome and gene expression levels: genomic DNA analysis identifies the genes that have been mutated, deleted or amplified, whereas RNA analysis reveals differences in transcription and RNA processing. cDNA microarrays were first developed to examine physiologically relevant gene expression patterns in yeast. Typically, a microarray consists of rows and rows of oligonucleotide strands or complementary DNAs (cDNAs) lined up in dots on a miniature silicon chip or glass slide. Oligonucleotides are synthesized in situ on the array and comprise short segments of DNA complementary to RNA transcripts of interest. Specifically for cancer research, several specialized [optimized] arrays relevant to particular tissues or physiological processes have been designed: mammary gland, lymphoid, apoptosis, angiogenesis, tumor invasion, tumor suppressor genes and the “oncochip”. Specific housekeeping genes are also incorporated to control for hybridization conditions and experimental variation. Typically microarray analysis requires that messenger RNA is extracted from tumor tissue, amplified and labeled with a fluorescent dye in a linear quantitative process. The labeled RNA is hybridized to the array in the presence of a reference RNA, which is labeled with a different fluorescent dye. After hybridization, the fluorescence from each spot on the array provides a measure of the relative abundance of a given transcript in the two RNA populations reflecting the relative expression level of its corresponding gene. In an embodiment, the expression levels are compared across many samples, both normal and pathological.

In an embodiment, a method for disease evaluation additionally comprises laser capture microdissection (LCM) technology. With this technique, malignant cells can be selectively dissected and captured so that only a morphologically homogeneous population of cells is investigated. However, inaccuracies still exist. A relatively small amount of material is recovered with the LCM process, thus recovered RNA does require amplification. Unbalanced amplification of transcripts could also lead to misinterpretation of cancer gene expression levels. Recent improvements in amplification technology are addressing this concern.

In an embodiment, the disease state of an individual may be evaluated through proteomic assays. Proteomics is the study of protein function. Proteins assemble themselves into networks through a variety of protein-protein interactions and posttranslational modifications. The amino acid sequence of a protein determines its 3-dimensional (3-D) shape. It is this shape and the surface presentation of nested amino acid motifs that enables highly selective lock-and-key recognition between protein partners in a communication circuit. Examples of these motifs include SH2, SH3, pleckstrin homology, and zinc fingers. These domains provide specific coupling points necessary for defined protein-protein interactions. Disruption of these coupling events may be the “functional” target of drug therapy. Examples of proteomic technologies include without limitation mass spectrometry (MS), 2-dimensional electrophoresis (2-DE), bead capture and micro-enzyme-linked immunosorbent assays (ELISA).

In an embodiment, the disease evaluation comprises 2D gel electrophoresis followed by protein identification using mass spectrometry. The state of the art 2D gel system can be loaded with a few nanograms of protein (traditional methodology requires about 100 cells) and separates thousands of protein spots. Samples are resolved in separate gels, making it difficult to accurately analyze and quantify individual proteins. Although a comparison of protein expression profiles from regular 2D gel electrophoresis can be carried out with the assistance of various software programs, it typically requires extensive computerized justification of 2D gel images so that two images can be superimposed and compared. These difficulties limit the speed and accuracy of quantitation of protein spots in 2D gel electrophoresis.

In an embodiment, 2-D gel electrophoresis is carried out using a differential in-gel electrophoresis (DIGE) technique. The concept of DIGE was originally published by Minden and colleagues and was subsequently developed at Amersham Biosciences. Typically, to analyze the samples in DIGE, two pools of protein extracts are labeled covalently with fluorescent cyanine dyes, Cy3 and Cy5, respectively. These labeled proteins are mixed and separated in the same 2D gel. The 2D gel patterns can be rapidly imaged by the fluorescence excitation of either Cy3 or Cy5 dyes. The amount of the dye is controlled in such a way that on average one protein molecule is labeled not more than once, and the minimum number of the molecules of each protein is labeled. A comparison of the resulting images allows quantitation of each protein spot. Because two pools of the proteins are separated in the same gel, those proteins existing in both pools will migrate to the same locations in the 2D gel, minimizing the reproducibility problem. Quantitation of the protein profile can be rapidly and accurately achieved based on the fluorescence intensity. With the improvement of the technology, it is possible to resolve 3 samples, separately labeled with Cy2, Cy3, and Cy5 on a single gel. This provides great versatility in comparing normal tissues with diseased tissues, tumor tissues before and at different time points after treatment, tumors of different individuals, or other combinations.

### EXAMPLES

The invention having been generally described, the following examples are given as particular embodiments of the invention and to demonstrate the practice and advantages thereof. It is to be understood that the examples are given by way of illustration and are not intended to limit the specification or the claims in any manner.

### Example 1

Eligible patients participating in phase I will be enrolled into one of 4 dose escalation cohorts: 3 patients each will be treated as per designated treatment dose-schedule. Patients 2 and 3 in each cohort may begin therapy after patient 1 has completed 2 weeks of therapy. Patient 1 in each subsequent cohort may begin therapy after all patients in the prior cohort have completed 2 weeks of therapy. If 1/3 patients in a cohort experiences dose limiting toxicity (DLT), 3 additional patients will be enrolled in that cohort. If >1/6 patients in a cohort experience DLT the prior cohort will be designated maximum tolerated dose (MTD); if <1/6 patients experiences DLT patients will accrue to the next cohort. If no DLT is documented in cohort 4 that dose schedule will be considered the effective clinical dose.

| | | | Agent X |
|---|---|---|---|
| | AVASTIN | TARCEVA | PO mg qd pm*, |
| Cohort | IV mg/m² q 3 weeks | PO mg qd am* | days 1-5/wk |
| 0 | 15 | 50 | x |
| 1 | 15 | 100 | x |
| 2 | 15 | 150 | x |
| 3 | 15 | 150 | 2x |

*Except for Cycles 1 and 2, Day 1 which TARCEVA and Agent X will both be taken in the morning to allow for pharmacokinetics.

### Example II

Phase II will enroll additional patients to a minimum of 13 patients at the MTD or effective clinical dose. Start AVASTIN+TARCEVA Cycle 1, Day 1 then add Agent X Cycle 2, Day 1.

As known to one of ordinary skill in the art, a DLT is indicted by grade 3 or 4 diarrhea (despite therapeutic intervention), rash, or nonhematologic toxicity, grade 4 hematologic toxicity, or treatment related death. Patients who experience a grade 3 or 4 hemorrhagic adverse event or grade 4 hypertension will be removed from trial. Other grade 3 or 4 serious adverse events related to study agents, following recovery to grade 1 after interruption, will either be treated at the prior cohort dose level or withdrawn if in cohort 0.

Toxicities will be graded and reported according to the NCI Common Toxicity Criteria for Adverse Events (CTCAE) Version 3.0 as linked in Appendix D. This document can also be downloaded from the Cancer Therapy Evaluation Program (CTEP) home page <http://ctep.info.nih.gov>.

While preferred embodiments of the invention have been shown and described, modifications thereof can be made by one skilled in the art without departing from the spirit and teachings of the invention. The embodiments described herein are exemplary only, and are not intended to be limiting. Many variations and modifications of the invention disclosed herein are possible and are within the scope of the invention. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the preferred embodiments of the present invention. The discussion of a reference herein is not an admission that it is prior art to the present invention, especially any reference that may have a publication date after the priority date of this application. The disclosures of all patents, patent applications, and publications cited herein are hereby incorporated by reference, to the extent that they provide exemplary, procedural or other details supplementary to those set forth herein.

What is claimed is:

1. A method of treatment comprising:
(a) evaluating the disease state of an individual using at least one assay;
(b) administering in amounts sufficient to treat said disease at least three agents wherein at least one agent is an inhibitor of the human epidermal receptor pathway, at least one agent is signal transduction inhibitor and at least one agent is an angiogenesis inhibitor; and
(c) reevaluating the disease state of said individual using at least one assay.

2. The method of claim 1 wherein the inhibitor of the human epidermal receptor pathway is a tyrosine kinase inhibitor.

3. The method of claim 1 wherein signal transduction inhibitor is an inhibitor of Akt, MAPK, JNK or combinations thereof.

4. The method of claim 1 wherein the signal transduction inhibitor is an alkylphospholipid.

5. The method of claim 1 wherein the angiogenesis inhibitor is an inhibitor of vascular endothelial growth factor.

6. The method of claim 1 wherein the agents comprise erlinotib, bevacizumab, an alkylphospholipid or combinations thereof.

7. The method of claim 1 further comprising an agent that is an oncogenic protein inhibitor.

8. The method of claim 7 wherein the oncogenic protein inhibitor is a tyrosine kinase inhibitor

9. The method of claim 1 wherein the oncogenic protein is BCR-Abl.

10. The method of claim 1 wherein the disease state of the individual is reevaluated, prior to administration of any or all of the agents, during administration of any or all of the agents, following administration of any or all of the agents or combinations thereof.

11. The method of claim 1 wherein the assays are quantitative, qualitative or combinations thereof.

12. The method of claim 1 wherein the assays comprise routine physical examinations, quality of life questionnaires, immunological assays, genomic assays, proteomic assays, or combinations thereof.

13. A composition comprising a first agent that is an inhibitor of the human epidemmal receptor pathway, a second agent that is an angiogenesis inhibitor and a third agent that is an inhibitor of a signal transduction pathway wherein said first agent, second agent, third agent and combinations thereof are present in amounts that when administered to individual in a diseased state are sufficient to treat said disease.

14. The composition of claim 13 further comprising an agent that is an oncogenic protein inhibitor.

15. The composition of claim 14 wherein the oncogenic protein comprises BCR-Abl.

16. The composition of claim 13 wherein the inhibitor of the human epidermal receptor pathway comprises a tyrosine kinase inhibitor.

17. The composition of claim 13 wherein the signal transduction inhibitor comprises an inhibitor of Akt, MAPK, JNK or combinations thereof.

18. The composition of claim 13 wherein the signal transduction inhibitor comprises an alkylphospholipid.

19. The composition of claim 17 wherein the angiogenesis inhibitor comprises an inhibitor of vascular endothelial growth factor.

20. The composition of claim 13 wherein the agents comprise erlinotib, bevacizumab, an alkylphospholipid or combinations thereof.

21. The composition of claim 13 further comprising an agent that is an oncogenic protein inhibitor.

22. The composition of claim 13 wherein the oncogenic protein inhibitor is a tyrosine kinase inhibitor

23. A method comprising administration of a composition comprising AVASTIN, TARCEVA and GLEEVAC.

24. A method comprising:
(a) evaluating the disease state of an individual using at least one quantitative and/or qualitative assay;
(b) administering in amounts sufficient to treat said disease at least three agents wherein at least at least one agent is signal transduction inhibitor; and
(c) reevaluating the disease state of said individual using quantitative and/or qualitative assays.
